SEQUENCE LISTING (142 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, synthetic construct
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, synthetic construct
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, synthetic construct [sequence omitted]

SEQ ID NO: 81 - protein, 108 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, synthetic construct [sequence omitted]

SEQ ID NO: 90 - protein, 119 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, synthetic construct [sequence omitted]

SEQ ID NO: 114 - protein, 215 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, synthetic construct
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, synthetic construct
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

SEQ ID NO: 141 - protein, 5 aa, Mus sp.
SSWIH

SEQ ID NO: 142 - protein, 17 aa, Mus sp.
EINPGNVRTNYNENFRN

(19) United States
(12) Patent Application Publication
(10) Pub. No.: US 2023/0049147 A1
(43) Pub. Date: Feb. 16, 2023

(54) ANTI-IL-36R ANTIBODIES FOR THE TREATMENT OF A FIBROTIC CONDITION

(71) Applicant: Boehringer Ingelheim International GmbH, Ingelheim am Rhein (DE)

(72) Inventors: Jay FINE, Mount Kisco, NY (US); Marc Kaestle, Schemmerhofen (DE); Janine Lamar, Ingelheim (DE); David Lamb, Mittelbiberach (DE); Meera Ramanujam, Danbury, CT (US); Sudha Visvanathan, Hartsdale, NY (US)

(21) Appl. No.: 17/819,327

(22) Filed: Aug. 12, 2022

Related U.S. Application Data
(60) Provisional application No. 63/232,780, filed on Aug. 13, 2021.
(60) Provisional application No. 63/335,256, filed on Apr. 27, 2022.

(51) Int. Cl. C07K 16/28; A61K 45/06; A61P 1/00; A61P 11/00; A61K 39/395

(52) U.S. Cl. CPC C07K 16/2866; A61K 45/06; A61P 1/00; A61P 11/00; A61K 39/3955; A61K 2039/505

(57) ABSTRACT

The present invention provides methods for treating, preventing or ameliorating a fibrotic condition. The methods of the present invention include administering to a patient suffering from a fibrotic condition a therapeutically effective amount an anti-interleukin-36 receptor (anti-IL-36R) antibody.

### SEQUENCE LISTING

The instant application contains a Sequence Listing which has been submitted in XML format via Patent Center and is hereby incorporated by reference in its entirety. Said XML copy, created on Aug. 1, 2022, is named 09-0724-US-3-2022-08-x-.xml and is 134 bytes in size.

### TECHNICAL FIELD OF THE INVENTION

The present invention related to the use of an anti-interleukin-36 receptor (anti-IL-36R) antibody for treating a fibrotic condition in a subject. For example, the invention relates to the use of anti-IL-36R antibody for treating a subject suffering from fibrostenotic crohn's disease (CD).

### BACKGROUND

All organs in the human body can be affected by fibrosis. The pathological fibrotic reactions could be systemic, such as in systemic sclerosis, affecting multiple organ system as well as affecting single organs, such as in the lung, liver, gastrointestinal (GI) tract, and kidney fibrosis. Nearly 45% of all deaths in the developed world are attributed to chronic fibroproliferative diseases.

One such fibrotic condition is fibrostenosing or fibrostenotic Crohn's disease (CD) that occurs in a subset of CD patients. CD is a lifelong inflammatory intestinal disease, which can impact any part of the GI tract. Pathogenesis of this disease involves a combination of genetic and environmental factors. Clinically, CD manifests with abdominal pain, chronic diarrhea, and weight loss. As the disease progresses, complications including strictures as well as abscesses and fistulae emerge. A majority of patients with CD will develop clinically apparent fibrostenosis, which can cause severe obstruction of the GI tract, necessitating endoscopic interventions or surgery.

According to latest national prevalence investigations, an estimated 3.1 million (1.3%) of US adults have been diagnosed with inflammatory bowel disease (IBD), including both CD and ulcerative colitis (UC). When CD patients progress to intestinal obstruction, all-cause surgical care would account for 44% of the lifetime costs, which is a significant socio-economic burden in developed countries. Despite the emergence of effective biological therapies, no available treatment can specifically target fibrosis, and the need for surgical intervention remained largely stable over time. Endoscopic intervention or surgery are the available approaches for CD-associated obstruction, which unfortunately only provide intermittent relief, since 70% of patients will have endoscopic evidence of disease recurrence at 1 year, while 40% of CD patients need further intervention at 4 years.

Thus, a need exists in the art for novel targeted therapies for the treatment and/or prevention of fibrotic conditions including fibrostenoic CD.

### SUMMARY OF THE INVENTION

The present invention addresses the above need by providing bio-therapeutics, in particular antibodies, which bind to IL-36R as a first- second-, third- or subsequent-line therapy for treating a fibrotic condition.

In one aspect, the present invention relates to a method for treating, preventing or ameliorating a fibrotic condition in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab.

A one aspect, the present invention relates to methods for the treatment of a fibrotic condition in an individual. This method includes administering to an individual having a fibrotic condition an effective amount of an anti-IL-36R antibody (as disclosed herein), wherein the fibrotic condition involves an internal organ or tissue, or ocular tissue, and the administration of the anti-IL-36R antibody is effective to treat the fibrotic condition. In an embodiment relating to this aspect, the anti-IL-36R antibody is spesolimab.

A number of fibrotic conditions can be treated in accordance with the present invention, including those involving internal organs such as lung, liver, kidney, heart, pancreas, gastrointestinal organs, and genitourinary organs; vascular tissue; and ocular tissue such as corneal tissue or retinal tissue.

Exemplary fibrotic conditions of the lung (i.e., pulmonary fibrosis) include, but are not limited to, idiopathic pulmonary upper lobe fibrosis (Amitani disease); familial pulmonary fibrosis; chronic hypersensitivity pneumonitis; sarcoidosis (Besnier-Boeck-Schaumann disease); pneumonitis or interstitial pneumonitis associated with collagenosis, pulmonary fibrosis secondary to e.g. lupus erythematodes, systemic scleroderma, rheumatoid arthritis, poly-myositis and dermatomysitis, idiopathic interstitial pneumonias, such as pulmonary lung fibrosis (IPF), idiopathic pulmonary upper lobe fibrosis (Amitani disease); non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hyper-sensitivity pneumonitis, or interstitial pneumonitis caused by different origins, e.g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-1-antitrypsin deficiency; non-specific interstitial pneumonia (“NSIP”); cryptogenic organizing pneumonia (“COP”); progressive massive fibrosis, a complication of coal worker's pneumoconiosis; scleroderma/systemic sclerosis; bronchiolitis obliterans-organizing pneumonia; pulmonary hypertension; pulmonary tuberculosis; silicosis; asbestosis; acute lung injury; and acute respiratory distress (“ARD”; including bacterial pneumonia induced, trauma-induced, and viral pneumonia-induced, ventilator-induced, non-pulmonary sepsis induced).

Exemplary fibrotic conditions of the liver (i.e., liver fibrosis) include, but are not limited to, liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis; infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections; and fibrosis secondary to radiation exposure.

Exemplary fibrotic conditions of the heart and/or pericardium (i.e., heart or pericardial fibrosis, or fibrosis of the associate vasculature) include, but are not limited to, endomyocardial fibrosis; cardiac allograft vasculopathy (“CAV”); myocardial infarction; atrial fibrosis; congestive heart failure; arterioclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis.

Exemplary fibrotic conditions of the kidney (i.e., kidney fibrosis) include, but are not limited to, glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubuloinsterstitial fibrosis; renal arteriosclerosis; Alport's syndrome; nephrotic syndrome; chronic renal failure; periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension.

Exemplary fibrotic conditions of the pancreas (i.e., pancreatic fibrosis) include, but are not limited to, stromal remodeling pancreatitis and stromal fibrosis.

Exemplary fibrotic conditions of the gastrointestinal tract (i.e., GI tract fibrosis) include, but are not limited to, fibrostenotic CD; collagenous colitis; colorectal fibrosis; villous atrophy; crypt hyperplasia; polyp formation; healing gastric ulcer; and microscopic colitis.

Exemplary fibrotic conditions of the eye include, but are not limited to, ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; corneal subepithelial fibrosis; and Grave's ophthalmopathy.

Additional fibrotic disorders or fibrosis resulting from any one of the aforementioned conditions include, but are not limited to, spinal cord injury/fibrosis or central nervous system fibrosis such as fibrosis after a stroke, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; and fibrosis incident to microbial infections (e.g., bacterial, viral, parasitic, fungal etc.).

In certain embodiments, the fibrotic condition is not coincidental to a cancerous condition, i.e., is not a cancerous proliferative disorder, and does not include idiopathic arthrofibrosis, or dermatological scarring.

In an embodiment relating to any of the above aspects, a second therapeutic agent is administered to the subject before, after, or concurrent with the anti-IL-36R antibody or an antigen-binding fragment thereof. In a related embodiment, the second therapeutic agent is selected from the group consisting of another IL-36R antagonist, an antifibrotic drug, an IgE inhibitor, a corticosteroid, NSAID, an IL-4R antagonist, a TNFα antagonist, and IFNγ.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In another embodiment relating to any of the above aspects and embodiments described herein, the anti-IL-36R antibody is administered in the range of about 0.001 to about 1000 mg to a subject suffering from a fibrotic condition.

In another embodiment relating to any of the above aspects and embodiments described herein, the anti-IL-36R antibody is administered in the range of about 1000 to about 2000 mg to a subject suffering from a fibrotic condition.

In another embodiment relating to any of the above aspects and embodiments described herein, the anti-IL-36R antibody is administered in the range of about 1000 to about 1500 mg to a subject suffering from a fibrotic condition.

In another embodiment relating to any of the above aspects and embodiments described herein, the anti-IL-36R antibody is administered in the range of about 1000 to about 1200 mg to a subject suffering from a fibrotic condition.

In another embodiment relating to any of the above aspects and embodiments described herein, the anti-IL-36R antibody is administered to about 1200 mg to a subject suffering from a fibrotic condition.

It will be understood that any of the herein disclosed methods, administration schemes and/or dosing regimens also equally apply to the use of any of the disclosed anti-IL-36R antibodies in such methods, administration schemes and/or dosing regimens: i.e. an anti-IL-36R antibody, as disclosed herein, for use in the treatment, prevention, reducing and/or amelioration of any of the disclosed diseases and/or conditions. In other words, the invention also provides for the use of an anti-IL-36R antibody, as disclosed herein, for the manufacture of a medicament for the treatment, prevention, reducing and/or amelioration of any of the disclosed diseases and/or conditions.

Additional features and advantages of the present invention will-become apparent from a review of the ensuing detailed description-set forth below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE FIGURES

The accompanying drawings, which are included to provide further understanding of the present invention and are incorporated in and constitute a part of this specification, illustrate aspects of the subject technology and together with the description serve to explain the principles of the present invention.

FIG. 1 shows that IL-36a and L-36g was measured in the bronchioalveolar lavage from healthy subjects and from patients with pulmonary fibrosis. A sub-set of patients (˜40%) demonstrated elevated concentrations of IL-36a and L-36g in the airways. Data is expressed as mean±SEM. Statistical differences tested with Mann-Whitnney test, p<0.05 is seen as statistical different.

FIG. 2 shows that IL-36 receptor was identified in cells from lung biopsies from healthy volunteers and patients with chronic hypersensitivity (cHP) by fluorescent in situ hybridization FISH. The percentage of cells positive in each tissue section was calculated. Data is expressed as mean±SD (n=4 healthy, n=3 cHP); Statistical differences tested with unpaired students t-test, * p<0.05.

FIG. 3 shows that A) Intratracheal instillation of IL-36g resulted in a disruption to the epithelial barrier after 4 hours as evidenced by the appearance of serum albumin in the airways. B) chronic overexpression of IL-36a and IL-36g in the lungs of mice also resulted in epithelial barrier disruption after 14 days as evidenced by increased surfactant protein-D in the blood, that was blocked with an anti-IL-36 receptor antibody. Data is expressed as mean±SD, Statistical differences tested with unpaired t-test (**p<0.01) or Ordinary-One-Way ANOVA test, using Sidak test for multiple comparison (* p<0.05).

FIG. 4 shows that Human dendritic cells were stimulated with depleted zymosan (a dectin-1 ligand) for 24 hours and next generation sequencing performed to identify deregulated genes. IL-36g was the second highest upregulated gene under these conditions.

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

In the following detailed description, numerous specific details are set forth to provide a full understanding of the present invention. It will be apparent, however, to one of ordinarily skilled in the art that the subject technology may be practiced without some of these specific details. In other instances, well-known structures and techniques have not been shown in detail so as not to obscure the present invention.

Unless defined otherwise, all technical and scientific terms used herein have the same meaning as commonly understood by one of ordinary skill in the art to which this invention belongs. Without wishing to be bound by this theory, the inventors believe that although the fibrotic conditions are a heterogeneous group of conditions, they have common features and overlapping pathophysiology which involves the IL-36 pathway. Ongoing inflammation, aberrant wound healing responses resulting in accelerated extracellular matrix deposition (ECM) and fibrosis as well as smooth muscle thickening are believed to drive the development of the fibrotic phenotype in fibrotic conditions.

For example, in fibrostenotic pathogenesis, the cross talk between inflammatory cells and myofibroblasts are thought to be central. The underlying mechanisms of fibrosis in CD are still unclear, which precludes the development of novel drugs. Inflammation-dependent and inflammation-independent mechanisms may both contribute to the initiation and progression of fibrosis. Intestinal mesenchymal cells, mainly fibroblasts, myofibroblasts, as well as smooth muscle cells (SMCs) are considered the main cell types contributing to the increased secretion of extracellular matrix (ECM). Accumulating ECM will ultimately lead to tissue fibrosis and stiffness, as well as intestinal strictures.

Recently generated data has shown a central role for IL-36 in mediating inflammatory and fibrotic mechanisms linked to fibrostenosis in CD. Overall, these data have demonstrated that (i) IL-36 activates immune cells such as dendritic cells and monocytes to produce proinflammatory mediators such as TNFα, IL-1β and IL-6 which are expressed in fibrostenotic tissues, (ii) IL-36 directly stimulates myofibroblasts to upregulate collagens and fibronectins, and to induce genes associated with epithelial-mesenchymal transition (EMT) and extracellular matrix degradation, suggesting a direct link with fundamental processes driving fibrostenosis pathology, (iii) the co-localization of IL-36α+inflammatory macrophages to intestinal myofibroblasts, indicates direct potential to induce collagen VI production and myofibroblast proliferation, which are the key hallmarks of fibrostenotic pathology, (iv) IL-36 pathway blockade therapeutically reverses established inflammation and intestinal fibrosis in chronic mouse colitis models.

Together, these findings support the hypothesis that an anti-IL-36R antibody (e.g., spesolimab) may be an effective agent for the treatment of a fibrotic condition (e.g., fibrostenotic CD).

Therefore, in one aspect, the present invention relates to a method for treating, preventing or ameliorating a fibrotic condition in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab.

Without wishing to be bound by this theory it is believed that anti-IL-36R antibodies or antigen-binding fragments thereof bind to human IL-36R and thus interfere with the binding of IL-36 agonists, and in doing so block at least partially the signaling cascade from the IL-36R to inflammatory mediators involved in fibrotic conditions. IL-36R is also known as IL-1 RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1 RAcP).

The anti-IL36R antibodies of the present invention are disclosed herein an in, for example, in U.S. Pat. No. 9,023,995, the entire content of which is incorporated herein by reference.

### Definitions

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

The term “subject” for purposes of treatment refers to any animal classified as a mammal, including humans, domesticated and farm animals, and zoo, sports, or pet animals, such as dogs, horses, cats, cows, and the like. Preferably, the mammal is human.

As used herein, the terms “treat”, “treating”, or the like, mean to alleviate symptoms, eliminate the causation of symptoms either on a temporary or permanent basis, or to prevent or slow the appearance of symptoms of the named disorder or condition. These terms are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-IL-36R antibody of the present invention, the result should be considered an effective treatment of the underlying disorder regardless of whether all the symptoms of the disorder are alleviated or not.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression.

Although any methods and materials similar or equivalent to those described herein can be used in the practice of the present invention, the preferred methods and materials are now described. All publications mentioned herein are incorporated herein by reference to describe in their entirety.

As mentioned earlier, a number of fibrotic conditions can be treated in accordance with the present invention, including those involving internal organs such as lung, liver, kidney, heart, pancreas, gastrointestinal organs, and genitourinary organs; vascular tissue; and ocular tissue such as corneal tissue or retinal tissue.

Exemplary fibrotic conditions of the lung (i.e., pulmonary fibrosis) include, but are not limited to, idiopathic pulmonary upper lobe fibrosis (Amitani disease); familial pulmonary fibrosis; chronic hypersensitivity pneumonitis; sarcoidosis (Besnier-Boeck-Schaumann disease); pneumonitis or interstitial pneumonitis associated with collagenosis, pulmonary fibrosis secondary to e.g. lupus erythematodes, systemic scleroderma, rheumatoid arthritis, poly-myositis and dermatomysitis, idiopathic interstitial pneumonias, such as idiopathic pulmonary fibrosis (IPF), idiopathic pulmonary upper lobe fibrosis (Amitani disease); non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous dis-ease, such as granulomatosis with polyangitis, Churg-Strauss syndrome, sarcoidosis, hypersensitivity pneumonitis, or interstitial pneumonitis caused by different origins, e.g. aspiration, inhalation of toxic gases, vapors, bronchitis or pneumonitis or interstitial pneumonitis caused by heart failure, X-rays, radiation, chemotherapy, M. boeck or sarcoidosis, granulo-matosis, cystic fibrosis or mucoviscidosis, or alpha-1-antitrypsin deficiency; non-specific interstitial pneumonia (“NSIP”); cryptogenic organizing pneumonia (“COP”); progressive massive fibrosis, a complication of coal worker's pneumoconiosis; scleroderma/systemic sclerosis; bronchiolitis obliterans-organizing pneumonia; pulmonary hypertension; pulmonary tuberculosis; silicosis; asbestosis; acute lung injury; and acute respiratory distress (“ARD”; including bacterial pneumonia induced, trauma-induced, and viral pneumonia-induced, ventilator-induced, non-pulmonary sepsis induced).

Exemplary fibrotic conditions of the liver (i.e., liver fibrosis) include, but are not limited to, liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis; infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections; and fibrosis secondary to radiation exposure.

Exemplary fibrotic conditions of the heart and/or pericardium (i.e., heart or pericardial fibrosis, or fibrosis of the associate vasculature) include, but are not limited to, endomyocardial fibrosis; cardiac allograft vasculopathy (“CAV”); myocardial infarction; atrial fibrosis; congestive heart failure; arterioclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis.

Exemplary fibrotic conditions of the kidney (i.e., kidney fibrosis) include, but are not limited to, glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubuloinsterstitial fibrosis; renal arteriosclerosis; Alport's syndrome; nephrotic syndrome; chronic renal failure; periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension

Exemplary fibrotic conditions of the pancreas (i.e., pancreatic fibrosis) include, but are not limited to, stromal remodeling pancreatitis and stromal fibrosis.

Exemplary fibrotic conditions of the gastrointestinal tract (i.e., GI tract fibrosis) include, but are not limited to, Crohn's disease; ulcerative colitis; collagenous colitis; colorectal fibrosis; villous atrophy; crypt hyperplasia; polyp formation; healing gastric ulcer; and microscopic colitis.

Exemplary fibrotic conditions of the eye include, but are not limited to, ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; corneal subepithelial fibrosis; and Grave's ophthalmopathy.

Additional fibrotic disorders or fibrosis resulting from any one of the aforementioned conditions include, but are not limited to, spinal cord injury/fibrosis or central nervous system fibrosis such as fibrosis after a stroke, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; and fibrosis incident to microbial infections (e.g., bacterial, viral, parasitic, fungal etc.).

In certain embodiments, the fibrotic condition is not coincidental to a cancerous condition, i.e., is not a cancerous proliferative disorder, and does not include idiopathic arthrofibrosis, or dermatological scarring.

As used herein, treatment of fibrosis or fibrotic conditions is meant to include disruption of the fibrotic processes so as to halt progression of the fibrotic condition, slow progression of the fibrotic condition, or cause regression of the fibrotic condition (i.e., improve the patient's state of health with respect to the degree of fibrosis in the affected tissue or organ). In certain embodiments, where treatment precedes onset of the fibrotic condition, i.e., treatment is performed prior to a known or an otherwise expected onset of fibrosis, then such treatment may include preventing development or onset of the fibrotic condition.

In one embodiment, the fibrotic condition is treated with an effective amount of an anti-IL-36R antibody of the present invention in the absence of any other agents. In other words, the invention consists of administering the anti-IL-36R antibody to the individual receiving such treatment, where such administering can be performed repeatedly over a period of time (including indefinitely) using an effective acceptable dosage schedule.

In another embodiment, the fibrotic condition is treated with an effective amount of an anti-IL-36R antibody in the presence of particular agents such as anti-proliferative agents used in the treatment of cancer. In other words, the invention consists essentially of administering the anti-IL-36R antibody to the individual receiving such treatment, where such administering can be performed repeatedly over a period of time (including indefinitely) using an effective acceptable dosage schedule. In this embodiment, additional agents can be administered to treat the fibrotic condition.

IL36R is a novel member of the IL1R family that forms a heterodimeric complex with the IL1R accessory protein (IL1 RAcp) and IL1 Rrp2 associated with epithelial mediated inflammation and barrier dysfunction. The heterodimeric IL36R system with stimulating (IL36α, IL36β, IL36γ) and inhibitory ligands (IL36Ra and IL38) shares a number of structural and functional similarities to other members of the IL1/ILR family, such as IL1, IL18 and IL33. All IL1 family members (IL1α, IL1β, IL18, IL36α, IL36β, IL36γ, and IL38) signal through a unique, cognate receptor protein which, upon ligand binding, recruits the common IL1 RAcP subunit and activates NFγB and MAP kinase pathways in receptor-positive cell types. As mentioned earlier, Recently generated data has shown a central role for IL-36 in mediating inflammatory and fibrotic mechanisms linked to fibrotic conditions such as fibrostenosis in CD (Foster et al., J Immunol. (2014) 192:6053-61; Vigne et al., Blood (2011) 118:5813-23; Scheibe et al., Gastroenterology (2019) 156(4):1082-1097)

Given the strong connection between the IL36 pathway and fibrotic conditions, without wishing to be bound by this theory, it is believed that IL36R biology contributes to pathophysiology of these conditions and hence blocking IL36R activation will be beneficial in patients suffering from a fibrotic condition.

Therefore, the present invention includes methods for treating a subject in need thereof, said method comprising administering a therapeutically effective amount of an anti-IL-36R antibody (disclosed herein) to the subject. As used herein, the expression “a subject in need thereof” means a human or a non-human animal that exhibits one or more symptoms of a fibrotic condition and/or one who has been diagnosed with a fibrotic condition such as fibrostenotic CD.

### Antibodies of the Present Invention

The anti-IL36R antibodies of the present invention are disclosed in U.S. Pat. No. 9,023,995 or WO2013/074569, the entire content of each of which is incorporated herein by reference.

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-36R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind IL-36R. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-36R, as used in the context of the present invention, includes antibodies that bind IL-36R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-36R may, however, have cross-reactivity to other antigens, such as IL-36R molecules from other (non-human) species.

In certain exemplary embodiments related to any aspects of the present invention, the anti-IL-36R antibody or antigen-binding fragment thereof that can be used in the context of the methods of the present invention includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3) or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

According to certain embodiments, the anti-IL-36R antibody or antigen-binding fragment thereof comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

### Therapeutic Uses of the Antibodies

The present invention provides anti-IL-36R antibodies and a pharmaceutical composition comprising an anti-IL-36R antibody for use in reducing, diminishing or blocking the IL-36 pathway which is useful for the treatment or prevention of a fibrotic condition in a subject, preferably a human patient.

In one aspect, the present invention relates to a method for treating, preventing or ameliorating a fibrotic condition in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab.

A one aspect, the present invention relates to methods for the treatment of a fibrotic condition in an individual. This method includes administering to an individual having a fibrotic condition an effective amount of an anti-IL-36R antibody (as disclosed herein), wherein the fibrotic condition involves an internal organ or tissue, or ocular tissue, and the administration of the anti-IL-36R antibody is effective to treat the fibrotic condition. In an embodiment relating to this aspect, the anti-IL-36R antibody is spesolimab.

A number of fibrotic conditions can be treated in accordance with the present invention, including those involving internal organs such as lung, liver, kidney, heart, pancreas, gastrointestinal organs, and genitourinary organs; vascular tissue; and ocular tissue such as corneal tissue or retinal tissue.

Exemplary fibrotic conditions of the lung (i.e., pulmonary fibrosis) include, but are not limited to, idiopathic pulmonary upper lobe fibrosis (Amitani disease); familial pulmonary fibrosis; chronic hypersensitivity pneumonitis; sarcoidosis (Besnier-Boeck-Schaumann disease); pneumonitis or interstitial pneumonitis associated with collagenosis, pulmonary fibrosis secondary to e.g. lupus erythematodes, systemic scleroderma, rheumatoid arthritis, poly-myositis and dermatomysitis, idiopathic interstitial pneumonias, such as idiopathic pulmonary fibrosis (IPF), idiopathic pulmonary upper lobe fibrosis (Amitani disease); non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hyper-sensitivity pneumonitis, or interstitial pneumonitis caused by different origins, e.g. aspiration, inhalation of toxic gases, vapors, bronchitis or pneumonitis or interstitial pneumonitis caused by heart failure, X-rays, radiation, chemotherapy, M. boeck or sarcoidosis, granulo-matosis, cystic fibrosis or mucoviscidosis, or alpha-1-antitrypsin deficiency; non-specific interstitial pneumonia (“NSIP”); cryptogenic organizing pneumonia (“COP”); progressive massive fibrosis, a complication of coal worker's pneumoconiosis; scleroderma/systemic sclerosis; bronchiolitis obliterans-organizing pneumonia; pulmonary hypertension; pulmonary tuberculosis; silicosis; asbestosis; acute lung injury; and acute respiratory distress (“ARD”; including bacterial pneumonia induced, trauma-induced, and viral pneumonia-induced, ventilator-induced, non-pulmonary sepsis induced).

Exemplary fibrotic conditions of the liver (i.e., liver fibrosis) include, but are not limited to, liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis; infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections; and fibrosis secondary to radiation exposure.

Exemplary fibrotic conditions of the heart and/or pericardium (i.e., heart or pericardial fibrosis, or fibrosis of the associate vasculature) include, but are not limited to, endomyocardial fibrosis; cardiac allograft vasculopathy (“CAV”); myocardial infarction; atrial fibrosis; congestive heart failure; arterioclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis.

Exemplary fibrotic conditions of the kidney (i.e., kidney fibrosis) include, but are not limited to, glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubuloinsterstitial fibrosis; renal arteriosclerosis; Alport's syndrome; nephrotic syndrome; chronic renal failure; periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension.

Exemplary fibrotic conditions of the pancreas (i.e., pancreatic fibrosis) include, but are not limited to, stromal remodeling pancreatitis and stromal fibrosis.

Exemplary fibrotic conditions of the gastrointestinal tract (i.e., GI tract fibrosis) include, but are not limited to, fibrostenotic CD; collagenous colitis; colorectal fibrosis; villous atrophy; crypt hyperplasia; polyp formation; healing gastric ulcer; and microscopic colitis.

Exemplary fibrotic conditions of the eye include, but are not limited to, ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; corneal subepithelial fibrosis; and Grave's ophthalmopathy.

Additional fibrotic disorders or fibrosis resulting from any one of the aforementioned conditions include, but are not limited to, spinal cord injury/fibrosis or central nervous system fibrosis such as fibrosis after a stroke, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; and fibrosis incident to microbial infections (e.g., bacterial, viral, parasitic, fungal etc.).

In certain embodiments, the fibrotic condition is not coincidental to a cancerous condition, i.e., is not a cancerous proliferative disorder, and does not include idiopathic arthrofibrosis, or dermatological scarring.

In an embodiment relating to any of the above aspects, a second therapeutic agent is administered to the subject before, after, or concurrent with the anti-IL-36R antibody or an antigen-binding fragment thereof. In a related embodiment, the second therapeutic agent is selected from the group consisting of another IL-36R antagonist, an antifibrotic drug, an IgE inhibitor, a corticosteroid, NSAID, an IL-4R antagonist, a TNFα antagonist, and IFNγ.

As used herein, treatment of fibrosis or fibrotic conditions is meant to include disruption of the fibrotic processes so as to halt progression of the fibrotic condition, slow progression of the fibrotic condition, or cause regression of the fibrotic condition (i.e., improve the patient's state of health with respect to the degree of fibrosis in the affected tissue or organ). In certain embodiments, where treatment precedes onset of the fibrotic condition, i.e., treatment is performed prior to a known or an otherwise expected onset of fibrosis, then such treatment may include preventing development or onset of the fibrotic condition.

In an embodiment relating to any of the above aspects or embodiments, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects or embodiments, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects or embodiments, the anti-IL-36R antibody includes:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects or embodiments, the anti-IL-36R antibody includes:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects or embodiments, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the above aspects or embodiments described herein, the anti-IL-36R antibody is administered in the range of 0.001 to 1000 mg to a subject suffering from a fibrotic condition.

In another embodiment relating to any of the above aspects and embodiments described herein, the anti-IL-36R antibody is administered in the range of about 1000 to about 2000 mg to a subject suffering from a fibrotic condition.

In another embodiment relating to any of the above aspects and embodiments described herein, the anti-IL-36R antibody is administered in the range of about 1000 to about 1500 mg to a subject suffering from a fibrotic condition.

In another embodiment relating to any of the above aspects and embodiments described herein, the anti-IL-36R antibody is administered in the range of about 1000 to about 1200 mg to a subject suffering from a fibrotic condition.

In another embodiment relating to any of the above aspects and embodiments described herein, the anti-IL-36R antibody is administered to about 1200 mg to a subject suffering from a fibrotic condition.

In a preferred embodiment relating to any of the above aspects or embodiments described herein, the fibrotic condition is fibrostenotic CD.

In another embodiment relating to any of the above aspects or embodiments described herein, the anti-IL-36R antibody is spesolimab.

In another embodiment relating to any of the above aspects or embodiments described herein, the therapeutic effective amount of the anti-interleukin-36 receptor (anti-IL-36R) antibody is in the range of about 0.001 to about 1000 mg.

In another embodiment relating to any of the above aspects and embodiments described herein, the anti-IL-36R antibody is administered in the range of about 1000 to about 2000 mg to a subject suffering from a fibrotic condition.

In another embodiment relating to any of the above aspects and embodiments described herein, the anti-IL-36R antibody is administered in the range of about 1000 to about 1500 mg to a subject suffering from a fibrotic condition.

In another embodiment relating to any of the above aspects and embodiments described herein, the anti-IL-36R antibody is administered in the range of about 1000 to about 1200 mg to a subject suffering from a fibrotic condition.

In another embodiment relating to any of the above aspects and embodiments described herein, the anti-IL-36R antibody is administered to about 1200 mg to a subject suffering from a fibrotic condition.

In another embodiment relating to any of the above aspects or embodiments described herein, a second therapeutic agent is administered to the subject before, after, or concurrent with the anti-IL-36R antibody.

In another embodiment relating to any of the above aspects or embodiments described herein, the second therapeutic agent is selected from the group consisting of another IL-36R antagonist, an antifibrotic drug, an IgE inhibitor, a corticosteroid, NSAID, an IL-4R antagonist, a TNFα antagonist, and IFNγ.

### Pharmaceutical Composition

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Sience and Practice of Phannacy, 19th Edition, Gennaro, Ed., Mack Publishing Co., Easton, Pa. 1995 which provides a compendium of formulation techniques as are generally known to practitioners.

A pharmaceutical composition comprising an anti-IL-36R monoclonal antibody of the present invention can be administered to a subject suffering from a fibrotic condition as described herein using standard administration techniques including oral, intravenous, intraperitoneal, subcutaneous, pulmonary, transdermal, intramuscular, intranasal, buccal, sublingual, or suppository administration.

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred.

The pharmaceutical composition typically must be sterile and stable under the conditions of manufacture arid storage in the container provided, including e.g., a sealed vial or syringe. Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrdse solution and Hank's solution and a therapeutically effective dose, (e.g., 1 to 100 mg/mL or more) of antibody concentration. Dose may vary depending on the type and severity of the disease and upon many factors, including the patient's size, body surface area, age, the particular compound to be administered, sex, time and route of administration, general health, and other drugs being administered concurrently. A typical dose can be, for example, in the range of 0.001 to 2000 mg; however, doses below or above this exemplary range are envisioned, especially considering the aforementioned factors.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL-36R antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

The following examples are intended to further illustrate certain preferred embodiments of the disclosure and are not limiting in nature. Those skilled in the art will recognize or be able to ascertain numerous equivalents to the specific substances and procedures described herein.

### Example 1: Treating Patients with a Fibrotic Condition

In this example, an anti-IL36R antibody of the present invention is used to treat patients suffering from a fibrotic condition.

Following the administration of the anti-IL-36R antibody, safety and efficacy assessments reveal the following: At least one patient with a fibrotic condition who receives a therapeutic effective amount of an anti-IL-36R antibody of the present invention shows improvements over baseline (immediately before the administration), or as compared to a patient with a similar condition who receives placebo.

### Example 2: Treating Patients with a Fibrostenotic CD

In this example, an anti-IL36R antibody of the present invention is used to treat patients with fibrostenotic CD.

The incidence and prevalence of Crohn's disease (CD) is rising (Molodecky N, et al. Incidence and prevalence of the inflammatory bowel diseases with time, based on systematic review. Gastroenterology 2012; 142:46-54). Despite up to 50% of patients with CD experiencing clinically significant intestinal fibrostenosis⋅(Cosnes J, et al. Long-term evolution of disease behavior of Crohn's disease. Inflamm Bowel Dis 2002; 8:244-250), there are currently no approved therapies to arrest or reverse accumulated bowel damage, specifically fibrostenosis. Furthermore, a lack of validated outcome measures for use in clinical trials has hindered drug development for this indication. Recent evidence indicates that interleukin 36 (IL-36), and the corresponding receptor (IL-36R), play an important role in the pathogenesis of fibrosis in multiple tissues, with inhibition of the IL-36 pathway shown to reduce fibrosis in preclinical models of chronic intestinal inflammation (Sheibe K, et al. Inhibiting Interleukin 36 Receptor signalling reduces fibrosis in mice with chronic intestinal inflammation. Gastroenterology 2019; 156:1082-1097).

A multicentre, double-blind, randomised, placebo-controlled, Phase IIa study investigated whether the anti-IL-36R antibody spesolimab can decrease symptomatic small bowel obstruction, and delay or reverse progression of fibrostenosis in patients who present with small bowel obstruction due to fibrostenotic CD.

The study objective was to evaluate the safety and efficacy of an antifibrotic agent for fibrostenosing small bowel Crohn's disease, using novel endpoints developed by the Stenosis Therapy and Anti-Fibrotic Research (STAR) consortium.

Eligible patients aged 18-75 years with symptoms of obstruction due to fibrostenotic CD, and a maximum of two terminal ileal strictures (diagnosed by one of the following: computed tomography, magnetic resonance imaging, computed tomography enterography or magnetic resonance enterography) first undergo optimization of anti-inflammatory therapy. In addition to any background biologic therapy they may receive, clinically responding patients are randomised 1:1 to receive either a 1200 mg intravenous dose of spesolimab or placebo. Randomisation is stratified according to the number of previously failed biologic therapies (0 or 1 vs >1) and stenosis length (5 cm vs >5 cm). Patients are assessed using magnetic resonance enterography, endoscopy and biopsy at Day 1, Week 24, and Week 48.

The primary endpoints assess the superiority of spesolimab versus placebo on one of the following: the proportion of patients with maintained symptomatic stenosis response (SSR) (defined as 7 days average score for both abdominal pain after eating <2, and amount or types of food limitation <2) at Week 48; and the proportion of patients with radiographic stenosis response (RSR) (defined as stricture shortening, or decrease in maximum associated small bowel dilation; either of which without new penetrating complications or stricture) at Week 48. An interim analysis to assess early time to treatment effect will be carried out when all patients have completed at least 24 weeks of treatment.

In alignment, the study's secondary endpoints are the proportion of patients with maintained SSR at Week 24, and the proportion of patients with maintained RSR at Week 24. Further objectives and endpoints are safety and tolerability of spesolimab; pharmacokinetic and antidrug antibody profiles of spesolimab; exploration of disease and IL-36 pathway-specific biomarkers; assessment of patient-reported outcomes; evaluation of psychometric changes; and time to relapse and/or surgery.

Following the administration of the anti-IL-36R antibody, safety and efficacy assessments reveal the following: At least one patient with a fibrostenotic CD who receives a therapeutic effective amount of an anti-IL-36R antibody of the present invention shows improvements over baseline (immediately before the administration), or as compared to a patient with a similar condition who receives placebo.

### Example 3: A Study to Test Whether Spesolimab Helps People with Crohn's Disease Who have Symptoms of Bowel Obstruction

Purpose: This study is open to adults, between 18 and 75 years of age, who have narrowings in the small bowel (strictures) due to fibrostenotic Crohn's disease. Strictures can lead to bowel obstruction (blockage). People whose symptoms got worse because of strictures can join the study. Participants get standard treatment for Crohn's disease and the strictures. The purpose of the study is to test whether the strictures improve further when treated with a medicine called spesolimab added to standard treatment.

Participants are in the study for about 1 year and 4 months. In the first 3 months, participants get standard treatment only. After 3 months, participants whose condition improved are put into 2 groups randomly, which means by chance. One group gets spesolimab added to their standard treatment. The other group gets placebo added to their standard treatment. Both spesolimab and placebo are given as infusions into a vein. Placebo infusions look like spesolimab infusions but do not contain any medicine. For the first 2 months, participants get the infusions every month. Thereafter, participants get the infusions every 2 months.

During the study, participants have about 11 visits to the study site. The doctors regularly check participants' health and take note of any unwanted effects. At 3 of the visits, doctors take images of the bowel using Magnetic Resonance Imaging and with an endoscope. At these visits, the doctors also take a small sample of bowel tissue (biopsy). The participants note their symptoms of Crohn's disease and how the symptoms affect daily life in an electronic diary. At the end of the study, results from the diaries and bowel imaging are compared between the spesolimab group and the placebo group.

Primary Outcome Measures: (1) Proportion of patients with maintained Symptomatic Stenosis Response [Time Frame: at Week 48]. No symptomatic relapse until Week 48. (2) Proportion of patients with Radiographic Stenosis Response [Time Frame: at Week 48]. Magnetic Resonance (Enterography) (MRE) stricture improvement.

Secondary Outcome Measures: (1) Proportion of patients with maintained Symptomatic Stenosis Response [Time Frame: at Week 24]. No symptomatic relapse until Week 24. (2) Proportion of patients with Radiographic Stenosis Response [Time Frame: at Week 24]. Magnetic Resonance (Enterography) (MRE) stricture improvement.

### Example 4: Treating Patients with Chronic Hypersensitivity or Other Inflammatory-Related Interstitial Lung Disease

Interleukin (IL)-36 is an inflammatory cytokine that activates the adaptor protein myeloid differentiated protein 88 (MyD88), mitogen-activated protein kinase (MAPK), and nuclear factor-kappa B (NF-κB) signalling pathways. These pathways facilitate the regulation of pro-inflammatory genes involved in immune cell activation, antigen presentation, and pro-inflammatory factor production. It is hypothesized that IL-36 is the inflammatory driver in inflammation associated ILDs, such as chronic hypersensitivity pneumonitis (cHP), SSc-ILD, RA-ILD and sarcoid-ILD and furthermore that this inflammation promotes lung epithelial damage and dysfunction and that it is the inappropriate repair of this damage that gives rise to fibrosis.

Upregulation of the IL-36 gene has been demonstrated in the lungs of a subset of patients with the most severe pulmonary fibrosis [1].

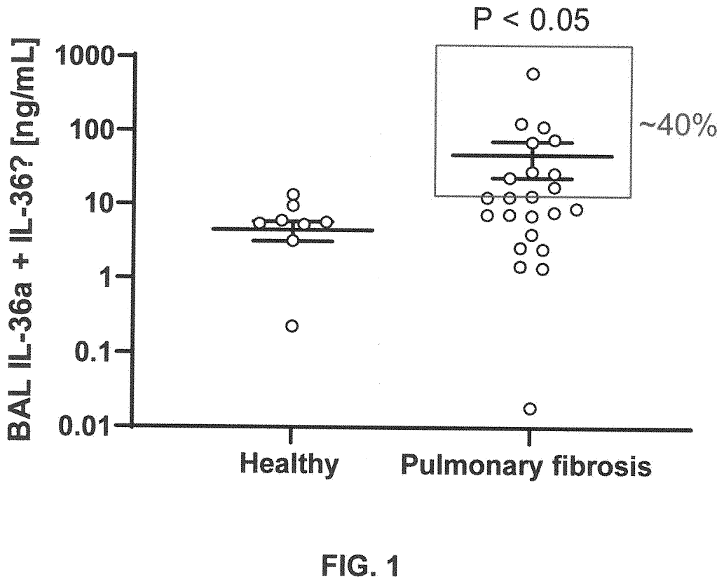

Bronchoalveolar lavage (BAL) samples from healthy individuals and patients with pulmonary fibrosis were centrifuged and supernatants were used for IL-36a and IL-36γ quantification by ELISA (R&D Systems, Cat. #: DY1078-05 and DY2320-05 respectively). Healthy individuals were defined as those samples which had no pulmonary fibrosis. Pulmonary fibrosis was assessed and defined by a high-resolution computer tomograph (HRCT) scan. To generate a value of total IL-36 burden, the IL-36α and IL-36γ were add together and given as II-36α+IL-36γ in pg/ml. FIG. 1 shows that the total IL-36 burden is significantly higher in the lung of patients with pulmonary fibrosis compared to those without pulmonary fibrosis.

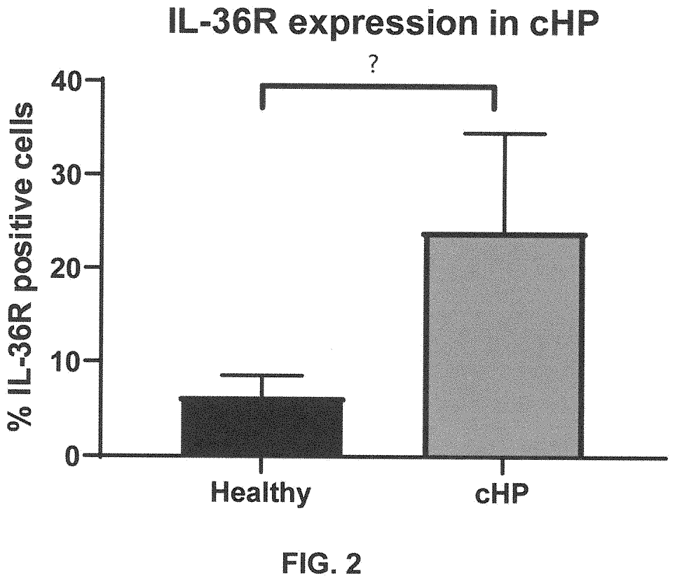

Lung biopsies from healthy individuals and patients with chronic hypersensitivity pneumonitis were paraffin embedded and 5 μm sections prepared. In situ hybridization for IL-36 receptor was performed using the Affymetrix QuantiGeneViewRNA assay. Paraffin sections were deparaffinized, boiled in pretreatment solution (Affymetrix, Santa Clara, Calif., USA) and digested with proteinase K. Sections were incubated with probe sets to detect IL-36 receptor at 40° C. The signal was amplified with Pre-Amp and Amp solutions (Affymetrix) and then developed with fast red substrate. The slides were counterstained with DAPI, mounted with Fluoromount (Sigma, St. Louis, Mo., USA) and scanned on a VS1200Olympus Slide Scanner, where TRITC channels were used to view IL-36 receptor. Dual-positive cells were then counted and presented as cells/mm² of tissue. FIG. 2 shows that IL-36 receptor is upregulated in the lungs of patients with cHP.

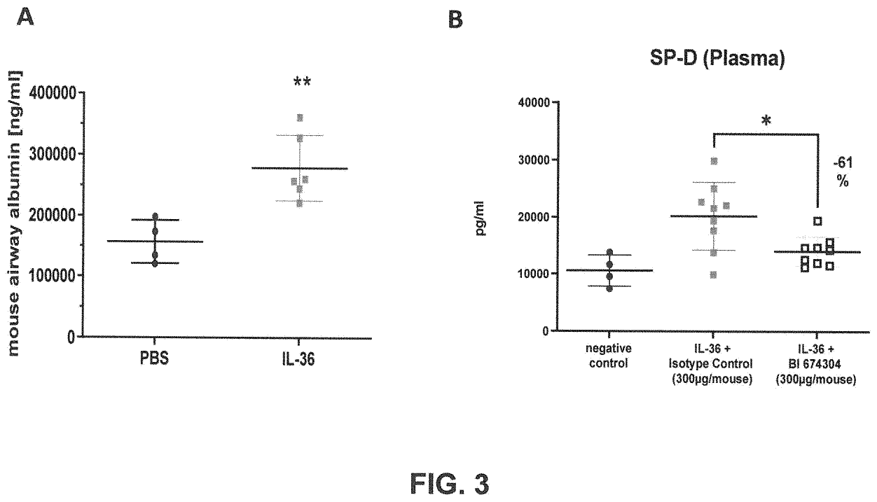

Mice were briefly anaesthetized with isoflurane and 1 μg recombinant IL-36g was instilled directly into the trachea. Twenty-four hours later, animals were euthanized and the lungs lavaged. Serum albumen was measured in the lavage fluid as a measure of epithelial barrier integrity. FIG. 3a shows that acute IL-36g instillation into mouse lungs promotes epithelial barrier dysfunction.

IL-36a and IL-36g were upregulated chronically in the lungs of mice using an adeno-associated virus (AAV) overexpression system. A 1:1 mixture of AAV6.2— an AAV with a high tropism for lung epithelial cells—containing DNA that encodes either IL-36a or IL-36g was instilled into the lungs of mice under transient anaesthesia with isoflurane. After 14 days, blood was drawn to measure plasma levels of surfactant protein-D (SP-D) an independent clinical predictor of pulmonary fibrosis progression [2]. FIG. 3b shows that chronic overexpression of IL-36a and IL-36g in the lungs of mice increases plasma SP-D that can be inhibited by an IL-36R blocking antibody.

Fungal infection is strongly linked aetiologically with cHP. In a mouse model of fugal extract-induced cHP, constitutive knockout of dectin-1 receptor signalling completely blocked the pulmonary inflammatory response [3].

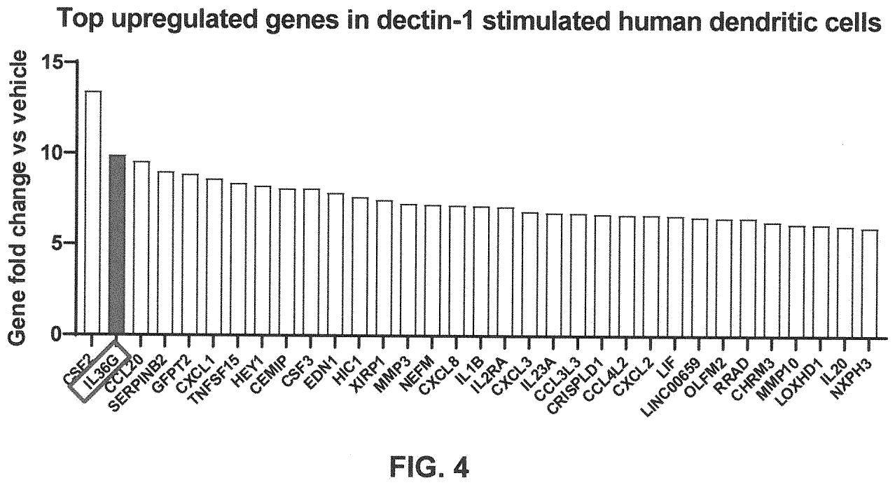

Human dendritic cells were derived from peripheral blood monocytes by differentiation using 100 ng/mL recombinant human interleukin-4 (IL-4) and ganulocyte-macrophage colony stimulating factor (GM-CSF) for 5 days and then 10 ng/mL human recombinant interleukin-1 beta (IL-1b), 20 ng/mL interleukin-6 (IL-6) and 20 ng/mL tumour necrosis factor-alpha (TNFa) for a further 2 days. Dendritic cells were then stimulated with depleted zymosan, a dectin-1 ligan, for 24 hours. FIG. 4 shows the genes upregulated by dectin-1 stimulation. IL-36g is the second highest upregulated gene, indicating that fungal components strongly upregulate IL-36g.

While there is no animal model data to link cHP with IL-36R antagonism, there is clinical evidence, provided by this patent that IL-36 cytokines are increased in lungs of patients and cHP patients showing significant increased IL-36R expression. To support the claim that pharmacological antagonism with an IL-36R blocking antibody has beneficial outcomes for cHP patients, the authors are planning to conduct the following study soon: According to Higashino-Kameda et al. [3] a cHP phenotype will be induced in mice via an intranasally application of T. asahii antigens (TIMM1318) on three consecutive days at the beginning and one boost application after 14 days. Mice will be sacrificed 7 days after the last antigen application. In three different dosing groups an IL-36R mouse tool antibody will be injected 2-3 times a week to verify the beneficial effect of IL-36R antagonism on lung inflammation and lung inflammatory infiltrates. The expected outcome of this study is a dose dependent improvement by the IL-36R blocking antibody regarding lung inflammation (decrease in immune cells and inflammatory cytokines) as well as decreased lung epithelial damage (decrease in plasma SP-D levels). Furthermore, the authors are planning to conduct a further study according to the protocol described by Hasan et al [4] in which extracts from S.rectivirgula are applied intranasally on days 1, 2, 3, 8, 9, 10, 15, 16 and 17 and the mice euthanized on day 21. This protocol elicits a lung fibrosis phenotype. In three different dosing groups an IL-36R mouse tool antibody will be injected 2-3 times a week to verify the beneficial effect of IL-36R antagonism on pulmonary fibrosis.

Given the strong connection between the IL36 pathway and cHP, without wishing to be bound by this theory, it is believed that IL36R biology contributes to pathophysiology of these conditions and hence blocking IL36R activation will be beneficial in patients suffering from a cHP. Therefore, the present invention includes methods for treating a subject in need thereof, said method comprising administering a therapeutically effective amount of an anti-IL-36R antibody or an antigen binding fragment thereof to the subject. As used herein, the expression “a subject in need thereof” means a human or a non-human animal that exhibits one or more symptoms of a cHP, e.g., immune cell infiltration in the lung, shortage of breath, and/or who has been diagnosed with cHP condition.

### REFERENCES

1. Wang, Y. et al. Unsupervised gene expression analyses identify IPF-severity correlated signatures, associated genes and biomarkers. Bmc Pulm Med 17, 133 (2017).
2. Maher, T. M. et al. An epithelial biomarker signature for idiopathic pulmonary fibrosis: an analysis from the multicentre PROFILE cohort study. Lancet Respir Medicine 5, 946-955 (2017).
3. Higashino-Kameda, M. et al. A critical role of Dectin-1 in hypersensitivity pneumonitis. Inflamm Res 65, 235-244 (2016).
4. Hasan, S. A. et al. Role of IL-17A and neutrophils in fibrosis in experimental hypersensitivity pneumonitis. J Allergy Clin Immunol 131 6, 1663-1673 (2003).

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method for treating a fibrotic condition in a subject, comprising administering to the subject a therapeutically effective amount of an anti-interleukin-36 receptor (anti-IL-36R) antibody.

2. The method of claim 1, wherein the fibrotic condition is selected from the group consisting of wherein the fibrotic condition is selected from the group consisting of a fibrotic condition of lung, a fibrotic conditions of the liver, a fibrotic conditions of the heart and/or pericardium, a fibrotic conditions of the kidney, fibrotic conditions of the pancreas, a fibrotic conditions of the gastrointestinal tract, a fibrotic conditions of the eye, and a fibrotic condition associated with a disease or disorder.

3. The method of claim 2,
wherein the fibrotic condition of lung comprises pulmonary fibrosis comprising idiopathic pulmonary upper lobe fibrosis (Amitani disease); familial pulmonary fibrosis; chronic hypersensitivity pneumonitis; sarcoidosis (Besnier-Boeck-Schaumann disease); pneumonitis or interstitial pneumonitis associated with collagenosis, pulmonary fibrosis secondary to e.g. lupus erythematodes, systemic scleroderma, rheumatoid arthritis, poly-myositis and dermatomysitis, idiopathic interstitial pneumonias, such as idiopathic pulmonary fibrosis (IPF), idiopathic pulmonary upper lobe fibrosis (Amitani disease); non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchy-mal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous dis-ease, such as granulomatosis with polyangitis, Churg-Strauss syndrome, sarcoidosis, hyper-sensitivity pneumonitis, or interstitial pneumonitis caused by different origins, e.g. aspira-tion, inhalation of toxic gases, vapors, bronchitis or pneumonitis or interstitial pneumonitis caused by heart failure, X-rays, radiation, chemotherapy, M. boeck or sarcoidosis, granulo-matosis, cystic fibrosis or mucoviscidosis, or alpha-1-antitrypsin deficiency; non-specific interstitial pneumonia (NSIP); cryptogenic organizing pneumonia (COP); progressive massive fibrosis, a complication of coal worker's pneumoconiosis; scleroderma/systemic sclerosis; bronchiolitis obliterans-organizing pneumonia; pulmonary hypertension; pulmonary tuberculosis; silicosis; asbestosis; acute lung injury; and acute respiratory distress (“ARD”; including bacterial pneumonia induced, trauma-induced, or viral pneumonia-induced, ventilator-induced, non-pulmonary sepsis induced); wherein the fibrotic condition of the liver (i.e., liver fibrosis) comprises liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis; infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections; or fibrosis secondary to radiation exposure; or
wherein the fibrotic condition of the heart and/or pericardium (i.e., heart or pericardial fibrosis, or fibrosis of the associate vasculature) comprises endomyocardial fibrosis; cardiac allograft vasculopathy (CAV); myocardial infarction; atrial fibrosis; congestive heart failure; arterioclerosis; atherosclerosis; vascular stenosis; myocarditis; congestive cardiomyopathy; coronary infarcts; varicose veins; coronary artery stenosis and other post-ischemic conditions; or idiopathic retroperitoneal fibrosis;
wherein the fibrotic condition of the kidney (i.e., kidney fibrosis) comprises glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubuloinsterstitial fibrosis; renal arteriosclerosis; Alport's syndrome; nephrotic syndrome; chronic renal failure; periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease or renal hypertension; or
wherein the fibrotic condition of the pancreas (i.e., pancreatic fibrosis) comprises stromal remodeling pancreatitis or stromal fibrosis; or
wherein the fibrotic condition of the gastrointestinal tract (i.e., GI tract fibrosis) comprises fibrostenotic Crohn's disease; ulcerative colitis; collagenous colitis; colorectal fibrosis; villous atrophy; crypt hyperplasia; polyp formation; healing gastric ulcer; or microscopic colitis; or
wherein the fibrotic condition of the eye comprises ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; corneal subepithelial fibrosis; or Grave's ophthalmopathy; or
wherein the fibrotic condition associated with a disease or disorder comprises spinal cord injury/fibrosis or central nervous system fibrosis such as fibrosis after a stroke, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; fibrosis incident to microbial infections (e.g., bacterial, viral, parasitic, fungal etc.); fibrosis coincidental to cancerous condition, i.e., is not a cancerous proliferative disorder, and does not include idiopathic arthrofibrosis, or dermatological scarring.

4. The method of claim 1, wherein the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

5. The method of claim 1, wherein the anti-IL-36R antibody comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

6. The method of claim 1, wherein the anti-IL-36R antibody comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

7. The method of claim 1, wherein the anti-IL-36R antibody is spesolimab.

8. The method of claim 1, wherein the therapeutic effective amount of the anti-interleukin-36 receptor (anti-IL-36R) antibody is in the range of about 0.001 to about 1000 mg.

9. The method of claim 1, wherein the therapeutic effective amount of the anti-IL-36R antibody is in the range of 1000 to about 2000 mg.

10. The method of claim 1, wherein the therapeutic effective amount of the anti-IL-36R antibody is in the range of about 1000 to about 1500 mg.

11. The method of claim 1, wherein the therapeutic effective amount of the anti-IL-36R antibody is in the range of about 1000 to about 1200 mg.

12. The method of claim 1, wherein the therapeutic effective amount of the anti-IL-36R antibody is about 1200 mg.

13. The method of claim 1, wherein a second therapeutic agent is administered to the subject before, after, or concurrent with the anti-IL-36R antibody.

14. The method of claim 13, wherein the second therapeutic agent is selected from the group consisting of another IL-36R antagonist, an antifibrotic agent, an IgE inhibitor, a corticosteroid, NSAID, an IL-4R antagonist, a TNFα antagonist, and IFNγ.

15. The method of claim 3, wherein the fibrotic condition is fibrostenotic CD.

16. The method of claim 3, wherein the fibrotic condition is chronic hypersensitivity pneumonitis (cHP).

17. The method of claim 1, wherein the treatment comprises disruption of the fibrotic processes so as to halt progression of the fibrotic condition, slow progression of the fibrotic condition, or cause regression of the fibrotic condition, or improve the patient's state of health with respect to the degree of fibrosis in the affected tissue or organ.

18. The method of claim 1, where the treatment precedes onset of the fibrotic condition, or the treatment is performed prior to a known or an otherwise expected onset of fibrosis for preventing development or onset of the fibrotic condition.
